(19) United States
(12) Patent Application Publication
(10) Pub. No.: US 2008/0319204 A1
(43) Pub. Date: Dec. 25, 2008

(54) PROCESS FOR THE SYNTHESIS OF PROGESTERONE RECEPTOR MODULATORS

(75) Inventors: Yanzhong Wu, Bardonia, NY (US); Karen Sutherland, New City, NY (US); John Leo Considine, Bridgewater, NJ (US); Bogdan Kazimierz Wilk, New City, NY (US); Pierre Giguere, Laval (CA); Michael MacEwan, Monroe, NY (US); Archana Sharma, Congers, NY (US); Alexander Gontcharov, Rivervale, NJ (US)

Correspondence Address: HOWSON AND HOWSON/WYETH;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 12/120,959

(22) Filed: May 15, 2008

Related U.S. Application Data
(60) Provisional application No. 60/937,108, filed on Jun. 25, 2007.

(51) Int. Cl. C07D 209/96

(52) U.S. Cl. 548/411

(57) ABSTRACT

Processes for preparing substituted oxindole-2-ones, and specifically the following, are described, wherein R¹-R⁴, R⁶, and n are defined herein. The processes include reacting a first alkali metal hydroxide, a tetraalkyl ammonium salt, a benzonitrile, and R⁶X or XCH₂(CH₂)ₙX′, wherein R⁶ is C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, heterocyclic, or substituted heterocyclic, X and X′ are, independently, leaving groups, and n is 1 to 5; (ii) reacting the product of step (i) with a second alkali metal hydroxide at a temperature of at least about 60° C.; (iii) reacting the product of step (ii) with an alkali alkoxide at a temperature of at least about 140° C. to form an oxindol-2-one; (iv) brominating the oxindol-2-one; and (v) coupling the brominated oxindol-2-one with a coupling reagent.

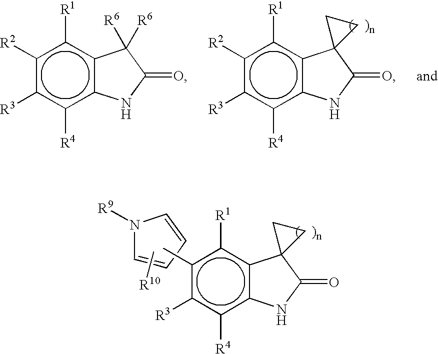

### CROSS-REFERENCE TO RELATED APPLICATIONS

This application claims the benefit of the priority of U.S. Provisional Patent Application No. 60/937,108, filed Jun. 25, 2007.

### BACKGROUND OF THE INVENTION

Cyclopropanation of phenylacetonitrile compounds has been performed using a base solution, such as a NaOH or KOH aqueous solution, a phase transfer catalyst, such as trimethylbenzylammonium chloride, and a dihaloalkane, such as 1,2-dibromoethane, 1,2-dichloroethane or 1,2-bromochloroethane (Singh et al. in U.S. Pat. No. 4,859,232 and Fedorynski et al. in Organic Preparation and Procedures Int., 27(3):355-359 (1995)). See, Scheme 1. However, the method of Singh required long reaction times (overnight) and excess 1,2-dibromomethane (3 mol equivalent) to provide only a moderate yield about 79%.

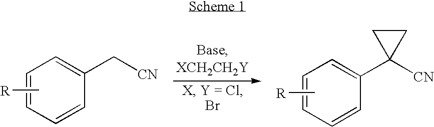

Hydrolysis of 1-arylcyclopropanecarbonitriles to their corresponding amides was also reported Singh et al. See, Scheme 2. However, this required long reaction times (2.5 days) and chromatography purification.

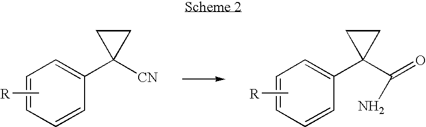

Hydrolysis of a nitrile to an amide with sodium hydroxide and hydrogen peroxide was described by Fieser et al. “Reagents for Organic Synthesis”, Wiley, New York, NY:469 (1967). However, the use of hydrogen peroxide prevented this procedure from being used on large scales due to safety concerns. Yet another method for converting nitriles to amides was described in Hall et al., J. Org. Chem., 41(23):3769-3770 (1976) in which potassium hydroxide in tert-butyl alcohol was utilized. However, this route was less desirable due to the high freezing point of tert-butyl alcohol.

Cyclization of amides, such as those illustrated in Scheme 3, can be performed using metal hydrides such as lithium hydrides in dimethylformamide (Fleming et al., J. Chem. Soc., Perkin Trans. 1: Org. and Bioorg. Chem. 2:349-59 (1986)). However, the combination of the metal hydride and DMF was explosive and thereby hazardous.

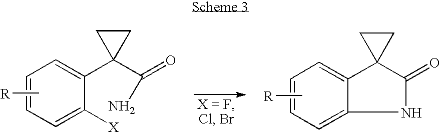

Bromination of 2-oxindoles with bromine in acetic acid is known (Adams, Bioorg. & Med. Chem. Lett. 13(18):3105-3110 (2003)). See, Scheme 4. However, such brominations resulted in region-isomers, i.e., 7-bromo isomers, and polybrominated products.

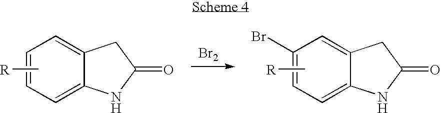

What are needed in the art are processes of preparing oxindolines that provide higher yields, minimize hazardous problems and provide clean product.

### SUMMARY OF THE INVENTION

In one aspect, processes for preparing substituted oxindole-2-ones are described.

In a further aspect, processes for preparing a substituted oxindol-2-one of the following structure, wherein R¹-R⁴ and R⁶ are defined below, are described.

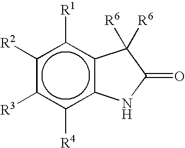

In another aspect, processes for preparing a substituted oxindol-2-one of the following structure, wherein R¹-R⁴ and n are defined below, are described.

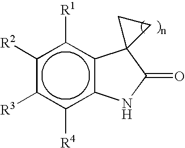

In a further aspect, processes for preparing a compound of the following structure,

wherein R¹, R³, R⁴, R⁹, R¹⁰, and n are defined below, are described.

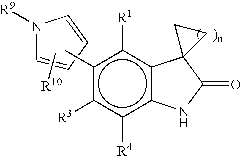

In still another aspect, processes for preparing the following compound are described.

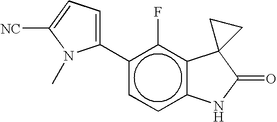

Other aspects and advantages of the invention will be readily apparent from the following detailed description of the invention.

### DETAILED DESCRIPTION OF THE INVENTION

The present invention provides processes for preparing substituted oxindol-2-ones. In one embodiment, the substituted oxindol-2-one is of the following structure:

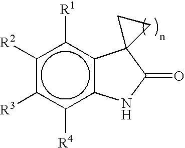

wherein, R¹, R³, and R⁴ are, independently, selected from among H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein the C₂ to C₆ alkynyl and substituted C₂ to C₆ alkynyl groups contain internal triple bonds; or R¹ and R³; R³ and R⁴; or R¹, R³, and R⁴ are fused to form (a) a 3 to 15 membered fully saturated, partially saturated, or fully unsaturated carbon-containing ring; or (b) a 3 to 15 membered heterocyclic ring containing in its backbone from 1 to 3 heteroatoms selected from among O, S, and NR¹¹; R² is C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, heterocyclic, or substituted heterocyclic; R⁵ is selected from among C₁ to C₆ alkyl and C₁ to C₆ substituted alkyl; and R¹¹ is absent, H, C₁ to C₆ alkyl, substituted C₁ to C₆ alkyl, aryl, or substituted aryl.

In another embodiment, the substituted oxindol-2-one is of the following structure, wherein R¹-R⁴ are defined above and R⁶ is C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, heterocyclic, or substituted heterocyclic.

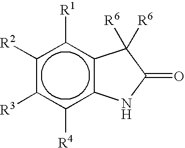

In a further embodiment, the substituted oxindol-2-one is of the following structure, wherein R¹, R³, R⁴, and n are defined above, R⁹ is selected from among C₁ to C₆ alkyl, substituted C₁ to C₆ alkyl, aryl, substituted aryl, heteroaryl, substituted heteroaryl, and COR^{A}; R^{A} is selected from among H, C₁ to C₆ alkyl, substituted C₁ to C₆ alkyl, C₁ to C₆ alkoxy, substituted C₁ to C₆ alkoxy, C₁ to C₆ aminoalkyl, and substituted C₁ to C₆ aminoalkyl; and R¹⁰ is selected from among H, OH, NH₂, CN, halogen, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₁ to C₆ alkoxy, substituted C₁ to C₆ alkoxy, C₁ to C₆ aminoalkyl, substituted C₁ to C₆ aminoalkyl, and COR^{A}.

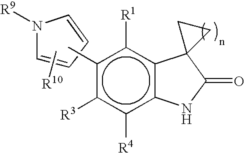

In still a further embodiment, the substituted oxindol-2-one is of the following structure, wherein R¹, R³, R⁴, R⁶, R⁹, and R¹⁰ are defined above.

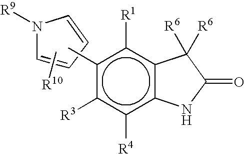

In yet another embodiment, the substituted oxindol-2-one is 5-(4′-fluoro-2′-oxo-1′,2′-dihydrospiro[cyclopropane-1,3′-indol]-5′-yl)-1-methyl-1H-pyrrole-2-carbonitrile.

The term “alkyl” is used herein to refer to both straight- and branched-chain saturated aliphatic hydrocarbon groups. In one embodiment, an alkyl group has 1 to about 10 carbon atoms (i.e., C₁, C₂, C₃, C₄, C₅ C₆, C₇, C₈, C₉, or C₁₀). In another embodiment, an alkyl group has 1 to about 6 carbon atoms (i.e., C₁, C₂, C₃, C₄, C₅ or C₆). In a further embodiment, an alkyl group has 1 to about 4 carbon atoms (i.e., C₁, C₂, C₃, or C₄).

The term “alkenyl” is used herein to refer to both straight- and branched-chain alkyl groups having one or more carbon-carbon double bonds. In one embodiment, an alkenyl group contains 2 to about 10 carbon atoms (i.e., C₂, C₃, C₄, C₅, C₆, C₇, C₈, C₉, or C₁₀). In another embodiment, an alkenyl group has 1 or 2 carbon-carbon double bonds and 2 to about 6 carbon atoms (i.e., C₂, C₃, C₄, C₅ or C₆).

The term “alkynyl” is used herein to refer to both straight- and branched-chain alkyl groups having one or more carbon-carbon triple bonds. In one embodiment, an alkynyl group has 2 to about 10 carbon atoms (i.e., C₂, C₃, C₄, C₅, C₆, C₇, C₈, C₉, or C₁₀). In another embodiment, an alkynyl group contains 1 or 2 carbon-carbon triple bonds and 2 to about 6 carbon atoms (i.e., C₂, C₃, C₄, C₅, or C₆).

The term “cycloalkyl” is used herein to refer to cyclic, saturated aliphatic hydrocarbon groups. The term cycloalkyl may include a single ring or two or more rings fused together to form a multicyclic ring structure. A cycloalkyl group may thereby include a ring system having 1 to about 5 rings. In one embodiment, a cycloalkyl group has 3 to about 14 carbon atoms (i.e., C₃, C₄, C₅, C₆, C₇, C₈, C₉, C₁₀, C₁₁, C₁₂, C₁₃, or C₁₄).

In another embodiment, a cycloalkyl group has 3 to about 6 carbon atoms (i.e., C₃, C₄, C₅ or C₆).

The terms “substituted alkyl”, “substituted alkenyl”, “substituted alkynyl”, and “substituted cycloalkyl” refer to alkyl, alkenyl, alkynyl, and cycloalkyl groups, respectively, having one or more substituents including, without limitation, hydrogen, halogen, CN, OH, NO₂, amino, aryl, heterocyclic, heteroaryl, alkoxy, aryloxy, alkylcarbonyl, alkylcarboxy, alkylamino, and arylthio.

The term “arylthio” as used herein refers to the S(aryl) group, where the point of attachment is through the sulfur-atom and the aryl group can be substituted as noted above.

The term “alkoxy” as used herein refers to the O(alkyl) group, where the point of attachment is through the oxygen-atom and the alkyl group can be substituted as noted above.

The term “aryloxy” as used herein refers to the O(aryl) group, where the point of attachment is through the oxygen-atom and the aryl group can be substituted as noted above.

The term “alkylcarbonyl” as used herein refers to the C(O)(alkyl) group, where the point of attachment is through the carbon-atom of the carbonyl moiety and the alkyl group can be substituted as noted above.

The term “alkylcarboxy” as used herein refers to the C(O)O(alkyl) group, where the point of attachment is through the carbon-atom of the carboxy moiety and the alkyl group can be substituted as noted above.

The term “alkylamino” as used herein refers to both secondary and tertiary amines where the point of attachment is through the nitrogen-atom and the alkyl groups can be substituted as noted above. The alkyl groups can be the same or different.

The term “halogen” as used herein refers to Cl, Br, F, or I groups.

The term “aryl” as used herein refers to an aromatic, carbocyclic system, e.g., of about 5 to 20 carbon atoms, which can include a single ring or multiple unsaturated rings fused or linked together where at least one part of the fused or linked rings forms the conjugated aromatic system. An aryl group may thereby include a ring system having 1 to about 5 rings. The aryl groups include, but are not limited to, phenyl, naphthyl, biphenyl, anthryl, tetrahydronaphthyl, phenanthryl, indene, benzonaphthyl, and fluorenyl.

The term “substituted aryl” refers to an aryl group which is substituted with one or more substituents including halogen, CN, OH, NO₂, amino, alkyl, cycloalkyl, alkenyl, alkynyl, alkoxy, C₁ to C₃ perfluoroalkyl, C₁ to C₃ perfluoroalkoxy, aryloxy, alkylcarbonyl, alkylcarboxy, —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, or heteroaryl, which groups can be substituted. Desirably, a substituted aryl group is substituted with 1 to about 4 substituents.

The term “heterocycle” or “heterocyclic” as used herein can be used interchangeably to refer to a stable, saturated or partially unsaturated 3- to 20-membered monocyclic or multicyclic heterocyclic ring. The heterocyclic ring has carbon atoms and one or more heteroatoms including nitrogen, oxygen, and sulfur atoms in its backbone. In one embodiment, the heterocyclic ring has 1 to about 4 heteroatoms in the backbone of the ring. When the heterocyclic ring contains nitrogen or sulfur atoms in the backbone of the ring, the nitrogen or sulfur atoms can be oxidized. Further, when the heterocyclic ring contains nitrogen atoms, the nitrogen atoms may optionally be substituted with H, C₁ to C₆ alkyl, substituted C₁ to C₆ alkyl, or CO₂ (C₁ to C₄ alkyl). The heterocyclic ring can be attached through a heteroatom or carbon atom provided the resultant heterocyclic ring structure is chemically stable. When the heterocyclic ring is a multicyclic ring, it may contain 2, 3, 4, or 5 rings.

A variety of heterocyclic groups are known in the art and include, without limitation, oxygen-containing rings, nitrogen-containing rings, sulfur-containing rings, mixed heteroatom-containing rings, fused heteroatom containing rings, and combinations thereof. Examples of heterocyclic groups include, without limitation, tetrahydrofuranyl, piperidinyl, 2-oxopiperidinyl, pyrrolidinyl, morpholinyl, thiamorpholinyl, thiamorpholinyl sulfoxide, pyranyl, pyronyl, dioxinyl, piperazinyl, dithiolyl, oxathiolyl, dioxazolyl, oxathiazolyl, oxazinyl, oxathiazinyl, benzopyranyl, benzoxazinyl and xanthenyl.

The term “heteroaryl” as used herein refers to a stable, aromatic 5- to 20-membered monocyclic or multicyclic heteroatom-containing ring. The heteroaryl ring has in its backbone carbon atoms and one or more heteroatoms including nitrogen, oxygen, and sulfur atoms. In one embodiment, the heteroaryl ring contains 1 to about 4 heteroatoms in the backbone of the ring. When the heteroaryl ring contains nitrogen or sulfur atoms in the backbone of the ring, the nitrogen or sulfur atoms can be oxidized. Further, when the heteroaryl ring contains nitrogen atoms, the nitrogen atoms may optionally be substituted with H, C₁ to C₆ alkyl, substituted C₁ to C₆ alkyl, or CO₂ (C₁ to C₄ alkyl). The heteroaryl ring can be attached through a heteroatom or carbon atom provided the resultant heterocyclic ring structure is chemically stable. When the heteroaryl ring is a multicyclic heteroatom-containing ring, it may contain 2, 3, 4, or 5 rings.

A variety of heteroaryl groups are known in the art and include, without limitation, oxygen-containing rings, nitrogen-containing rings, sulfur-containing rings, mixed heteroatom-containing rings, fused heteroatom containing rings, and combinations thereof. Examples of heteroaryl groups include, without limitation, furyl, pyrrolyl, pyrazolyl, imidazolyl, triazolyl, pyridyl, pyridazinyl, pyrimidinyl, pyrazinyl, triazinyl, azepinyl, thienyl, dithiolyl, oxathiolyl, oxazolyl, thiazolyl, oxadiazolyl, oxatriazolyl, oxepinyl, thiepinyl, diazepinyl, benzofuranyl, thionapthene, indolyl, benzazolyl, purindinyl, pyranopyrrolyl, isoindazolyl, indoxazinyl, benzoxazolyl, quinolinyl, isoquinolinyl, benzodiazonyl, napthylridinyl, benzothienyl, pyridopyridinyl, acridinyl, carbazolyl, and purinyl rings.

The term “substituted heterocycle” and “substituted heteroaryl” as used herein refers to a heterocycle or heteroaryl group having one or more substituents including halogen, CN, OH, NO₂, amino, alkyl, cycloalkyl, alkenyl, alkynyl, C₁ to C₃ perfluoroalkyl, C₁ to C₃ perfluoroalkoxy, alkoxy, aryloxy, alkylcarbonyl, alkylcarboxy, —C(NH₂)═N—OH, —SO₂—(C₁ to C₁₀ alkyl), —SO₂—(C₁ to C₁₀ substituted alkyl), —O—CH₂-aryl, alkylamino, arylthio, aryl, or heteroaryl, which groups may be optionally substituted. A substituted heterocycle or heteroaryl group may have 1, 2, 3, or 4 substituents.

The process described herein includes reacting a first alkali metal hydroxide, a tetraalkyl ammonium salt, a benzonitrile, and R⁶X or XCH₂(CH₂)ₙX′, wherein R⁶ is C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, heterocyclic, or substituted heterocyclic, X and X′ are leaving groups, and n is 1 to 5. X and X′ may be the same or may differ. In one example, XCH₂(CH₂)ₙX′ is dibromoethane. If R⁶X is utilized, at least 2 equivalents are added to the reaction mixture. In one example, about 2 to about 4 equivalents are R⁶X are utilized. Alternatively, if XCH₂(CH₂)ₙX′ is utilized, at least 1 equivalent is added to the reaction mixture. In one example, about 1 to about 3 equivalents of XCH₂(CH₂)ₙX′ are utilized.

One of skill in the art would readily be able to select a suitable first alkali metal hydroxide for use in the reaction. The first alkali metal hydroxide includes, without limitation, sodium hydroxide, potassium hydroxide, or lithium hydroxide. In one embodiment, the first alkali metal hydroxide is sodium hydroxide. Desirably, an excess of the first alkali metal hydroxide is utilized. The term “excess” as used herein refers to greater than 1 molar equivalent of a chemical compound. More desirably, about 2 to about 5 equivalents of the first alkali metal hydroxide are utilized. Most desirably, about 5 equivalents of the first alkali metal hydroxide are utilized.

The tetraalkyl ammonium salt may also be selected by one of skill in the art. Suitable tetraalkylammonium salts include, without limitation, tetrabutylammonium bromide. The amount of the tetralkylammonium salt may be determined by one of skill in the art. Typically, a catalytic amount of the tetraalkylammonium salt is utilized. The term “catalytic amount” as used herein refers to the minimum amount of a chemical compound, i.e., a catalyst, that is required to accelerate a reaction. However, equimolar or excess amounts of the tetraalkylammonium salt may be utilized. Desirably, about 0.04 equivalents of the tetraalkylammonium salt may be utilized.

The reaction is typically performed in an organic solvent which may be readily selected by one of skill in the art including, without limitation, N-methylpyrrolidone (NMP), N,N-dimethylacetamide, methyl t-butyl ether (MTBE), or combinations thereof. The temperature of the reaction mixture is typically maintained at about 20 to 65° C. Desirably, the temperature is maintained at about 35 to about 45° C. The time required to complete the reaction may easily be determined by one of skill in the art. Desirably, the reaction is performed for about 1 hour to about 24 hours. More desirably, the reaction is performed in about 2 hours.

After the reaction, the product is isolated from the reaction mixture using techniques in the art. In one example, the temperature is typically adjusted to about 45° C. and stirred, typically for about 2 hours. Water is slowly added to the solution, the solution cooled to about room temperature, i.e., about 20° C. to about 25° C., and an organic solvent added to the solution to extract the product. One of skill in the art would readily be able to select a suitable organic solvent for extraction and may include, without limitation, MTBE, toluene, ethyl acetate, t-amyl alcohol, or combinations thereof. Separation of the organic phase, concentration via vacuum distillation, dissolving the residue in a solvent, such as t-amyl alcohol or t-butyl alcohol, for removal of residual water and unreacted R⁶X or XCH₂(CH₂)ₙX′, BrCH₂CH₂Br, provides the product in the solvent which is directly used for next step.

A variety of benzonitrile compounds may be utilized in the reaction. In one embodiment, the benzonitrile is of the following structure, wherein R¹, R³, and R⁴ are defined above and LG is a leaving group. The term “leaving group” as used herein refers to a chemical moiety that is displaced from a first chemical compound upon reaction of the first chemical compound with a second chemical compound. Examples of suitable leaving groups include, without limitation, halogens such as chlorine or bromine or fluorine, tosylates, mesylates, triflates, and sulfonates.

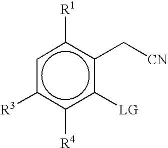

By doing so and in one embodiment, the product of step (i) is of the following structure, wherein R¹, R³, R⁴, R⁶, and LG are defined above.

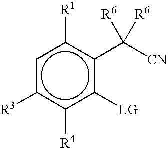

In another embodiment, the product of step (i) is of the following structure,

wherein R¹, R³, R⁴, n, and LG are defined herein.

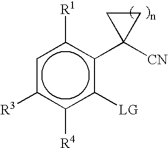

In still another embodiment, the product of step (i) is 4′-fluorospiro[cyclopropane-1,3′-indolin]-2′-one.

The product of step (i) is then reacted with a second alkali metal hydroxide at a temperature of at least about 60° C. to form an amide. The second alkali metal hydroxide may be selected by one of skill in the art including sodium hydroxide, potassium hydroxide, or lithium hydroxide. Desirably, the second alkali metal hydroxide is potassium hydroxide. In one embodiment, step (ii) is performed at a temperature of about 60 to about 100° C. In another embodiment, step (ii) is performed at a temperature of about 70° C.

The product, i.e., the amide, is then isolated from the reaction mixture using techniques known in the art. In one embodiment, the reaction mixture is cooled to a temperature of about 30° C., water is added, the mixture is stirred, desirably for about 15 minutes, the organic phase is isolated and concentrated by vacuum distillation, the residue chased in a solvent such as toluene, t-amyl alcohol, or a combination thereof, any residual water removed, and the residue then dissolved in a solvent which may selected by one of skill in the art including, without limitation, NMP or N,N-dimethylacetamide.

In one embodiment, the product of step (ii) is of the following structure, wherein R¹, R³, R⁴, R⁶, and LG are defined herein.

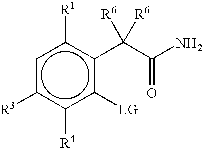

In another embodiment, the product of step (ii) is of the following structure, wherein R¹, R³, R⁴, n, and LG is defined herein.

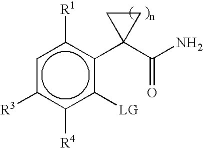

The amide is then reacted with an alkali alkoxide at a temperature of at least about 140° C. to form an oxindol-2-one. One of skill in the art would readily be able to select a suitable alkali alkoxide for use in the reaction including, without limitation, sodium t-pentoxide, sodium t-butoxide, or potassium t-butoxide. Desirably, the alkali alkoxide is sodium t-pentoxide. In one embodiment, the reaction is performed at a temperature of about 140° C. to about 180° C. In a further embodiment, the reaction is performed at a temperature of about 140° C. to about 160° C. In another embodiment, the reaction is performed at a temperature of about 145° C. Desirably, the reaction is stirred for about 1 to about 12 hours, depending on the alkali metal alkoxide utilized. In one example, the reaction mixture is stirred for about 4 to about 9 hours when the temperature is about 145° C. In another example, the reaction is stirred for about 1 to about 12 hours when a temperature of about 140 to about 160° C. is utilized. Upon completion, the reaction mixture may be worked-up using techniques known in the art. In one embodiment, the reaction mixture may be cooled to room temperature and slowly added to a cold acid solution over a period of about 1 hour while maintaining the temperature at about 10° C. to about 20° C. Desirably, the acid solution is a hydrochloric or sulfuric acid solution. The resultant mixture, which contains a precipitate and has a pH of about 1 to about 4, may be cooled to about 3° C. to about 7° C., stirred for about 1 hour, and filtered to provide the solid product. The solid may then be filtered using techniques in the art, washed with water, desirably washed 3 times with water, and dried at elevated temperatures of about 55° C. under reduced pressure.

In one embodiment, the oxindol-2-one is of the following structure, wherein R¹, R³, R⁴, and R⁶ are defined herein.

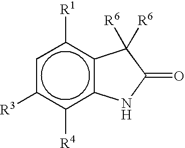

In another embodiment, the oxindol-2-one is of the following structure, wherein R¹, R³, R⁴, and n are defined herein.

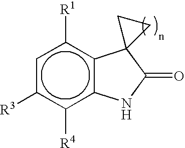

In a further embodiment, the oxindol-2-one is 4′-fluorospiro[cyclopropane-1,3′-indolin]-2′-one.

The oxindol-2-one is then brominated using techniques known to those of skill in the art including Adams et al., Bioorganic & Medicinal Chemistry Letters (2003), 13(18), 3105-3110 and Jiang et al., Faming Zhuanli Shenqing Gongkai Shuomingshu (2006), 25 pp., to form a brominated oxindol-2-one. The bromination is performed using a brominating agent that may be readily selected by one skilled in the art. Desirably, the bromination is performed using N-bromo-succinimide (NBS) or dibromo-dimethylhydantoin. More desirably, the bromination is performed using NBS. The reaction may be performed in a mixture of water and an organic solvent such as acetonitrile, acetic acid, or dichloromethane, among others. Desirably, the ratio of water to acetonitrile is about 0:10 to about 10:0. Desirably, the reaction is performed at a temperature of about 0° C. to about 30° C. More desirably, the reaction is performed at a temperature of about 0 to about 20° C. After addition of the brominating agent, the reaction is quenched using techniques known in the art, including the addition of water, among others. Desirably, about 3 parts of water, over the volume of the solution, is added. The mixture may then be cooled to a temperature of about 0° C. to about 6° C. and stirred, typically for about 30 minutes. The product may then be collected using filtration or the like, washed with water, and dried. Drying is typically performed at reduced pressures and at elevated temperatures. More desirably, drying is performed at about 55° C. for 18 hours at 0-10 mm Hg. By doing so, the brominated oxindol-2-one is prepared in a yield of at least 90%, 91%, 92%, 93%, 94%, 95%, 96%, 97%, 98%, 99% or 100%. The brominated oxindol-2-one may be used without further purification or may be purified using techniques in the art.

In one embodiment, the brominated oxindol-2-one is of the following structure, wherein R¹, R³, R⁴, and R⁶ are defined herein.

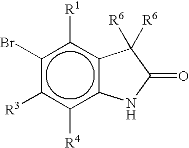

In another embodiment, the brominated oxindol-2-one is of the following structure, wherein R¹, R³, R⁴, and n are defined herein.

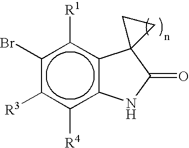

In a further embodiment, the brominated oxindol-2-one is 5′-bromo-4′-fluorospiro[cyclopropane-1,3′-indolin]-2′-one.

The brominated oxindol-2-one is then coupled with a coupling reagent using techniques known to those of skill in the art to form the substituted oxindol-2-one discussed above. In one embodiment, the procedure set forth in US Patent Application Publication No. US-2005-0272702-A1 is utilized to couple the brominated 2-oxindole with a coupling reagent. Desirably, the brominated 2-oxindole is coupled at the 5-position.

In one embodiment, the coupling reagent is R²D, wherein D is a leaving group and R² is C₁ to C₆ alkyl, substituted C₁ to C₆ alkyl, C₃ to C₈ cycloalkyl, substituted C₃ to C₈ cycloalkyl, aryl, substituted aryl, heteroaryl, or substituted heteroaryl. In a further embodiment, R² is aryl, substituted aryl, heteroaryl, or substituted heteroaryl. In another embodiment, the coupling reagent is an optionally substituted cyanopyrrole. In a further embodiment, the coupling reagent is [1,3,6,2]dioxazaborocan-2-yl-1-methyl-1H-pyrrole-2-carbonitrile.

Desirably, the coupling is performed in the presence of a palladium catalyst and a weak base. In one embodiment, the coupling is performed in the presence of a Pd(II) catalyst. In another embodiment, the coupling is performed in the presence of dichloro bis(triphenylphosphine) palladium (II) or tris(dibenzylideneacetone)dipalladium (Pd₂(dba)₃) in the presence of 1,1′-bis(diphenylphosphino)ferrocene (dppf) or diphenylphosphino phenylether (DPE). Desirably, the palladium catalyst is dichloro bis(triphenylphosphine) palladium (II). In one example, at least a 1% mol equivalent of the Pd catalyst as compared to the brominated oxindol-2-one is utilized. In another example, about 1 to about 10% mol equivalent of the Pd catalyst as compared to the brominated oxindol-2-one is utilized. The base is typically an inorganic base such as an alkali metal hydroxide, alkali metal carbonate, or alkali metal phosphate. In one example, the base is sodium hydroxide, potassium hydroxide, sodium hydrogen carbonate, potassium hydrogen carbonate, sodium carbonate, potassium carbonate, sodium phosphate, potassium phosphate, sodium hydrogen phosphate, or potassium hydrogen phosphate. In another example, the weak base is sodium or potassium carbonate. An equimolar or excess of the base is desirably utilized. In one embodiment, about 1.0 to about 2.0 equivalents of the weak base are utilized. In a further embodiment, about 1.2 equivalents of the weak base are utilized.

The coupling may be performed using any solvent, which can readily be determined by one of skill in the art. In one embodiment, the coupling is performed in acetonitrile, THF or 1,2-dimethoxyethane, optionally in the presence of water, e.g., about 1 to about 5 parts of water. In another embodiment, the coupling is performed in THF at a temperature of about 55° C. to about 66° C. In a further embodiment, the coupling is performed in THF at a temperature of about 66° C. The coupling is generally complete in about 2 to about 3 hours; however, the reaction is not so limited and may take less or more time to complete depending on the brominated oxindole and coupling reagents utilized.

The substituted oxindol-2-one may be isolated using techniques known to those skilled in the art including filtration, decanting, extraction, and distillation, among others. In one embodiment, the substituted oxindol-2-one is isolated from the coupling step. Typically, the substituted oxindol-2-one is extracted using an organic solvent. One of skill in the art would readily be able to select a suitable organic solvent that dissolves the substituted oxindol-2-one. In one embodiment, the substituted oxindol-2-one is dissolved in THF.

The palladium catalyst may then be removed using N-acetyl-L-cysteine as described in Christine (Adv. Synth. Catal. (2004) 346:889-900). Specifically, N-acetyl-L-cysteine may be added to the organic solvent which contains the substituted oxindol-2-one. However, the inventors found that by adding about 0.025 to about 0.50 equivalents of N-acetyl-L-cysteine to the organic layer containing the substituted oxindol-2-one, the residual palladium may be removed. Desirably, about 0.5 equivalents of N-acetyl-L-cysteine is added. The residual unsoluble palladium may then be removed using filtration techniques known to those of skill in the art. In one embodiment, the filtration is performed using a Celite® reagent/charco/Celite® reagent pad. In another embodiment, the organic layer is refluxed with the N-acetyl-cysteine solution for about 1 hour and then filtered through a Celite® reagent/charco/Celite® reagent pad.

Once filtered, the organic solvent may be removed from the solution using techniques known to those skilled in the art including, without limitation, reduced pressures, to provide a crude solid containing the substituted oxindol-2-one product. The crude solid may then be dissolved in an organic solvent, desirably 2-propanol at an elevated temperature, and the substituted oxindol-2-one recrystallized therefrom using techniques known to those in the art. Typically, the 2-propanol solution is concentrated and the recrystallized substituted oxindol-2-one crystallized therefrom upon cooling. The inventors found that recrystallization removed any further traces of the palladium catalyst and/or N-acetyl-L-cysteine reagent remaining from the previous steps.

The substituted oxindol-2-one collected from the recrystallization may then be further purified using techniques known in the art. In one embodiment, the recrystallized substituted oxindol-2-one is washed with cold 2-propanol, dried at a temperature of about 45° C. to about 55° C. under reduced pressures, slurried in ethyl acetate at a temperature of about 45° C. to about 50° C. for about 2 to about 3 hours, cooled to about 5° C. to about 10° C., the solid collected using filtration, the solid washed with cold ethyl acetate, and the solid dried using reduced pressures to provide the pure substituted oxindol-2-one. Desirably, the substituted oxindol-2-one is isolated at greater than 95%, 96%, 97%, 98%, or 99% HPLC purity.

In one embodiment, a process for preparing a substituted oxindol-2-one is provided and includes (i) reacting a first alkali metal hydroxide, a tetraalkylammonium salt, a benzonitrile, and R⁶X, wherein R⁶ is C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, heterocyclic, or substituted heterocyclic and X is a leaving group; (ii) reacting the product of step (i) with a second alkali metal hydroxide at a temperature of at least about 60° C.; (iii) reacting the product of step (ii) with an alkali alkoxide at a temperature of at least about 140° C. to form an oxindol-2-one; (iv) brominating the oxindol-2-one; and (v) coupling the brominated oxindol-2-one with a coupling reagent. See, Scheme 5.

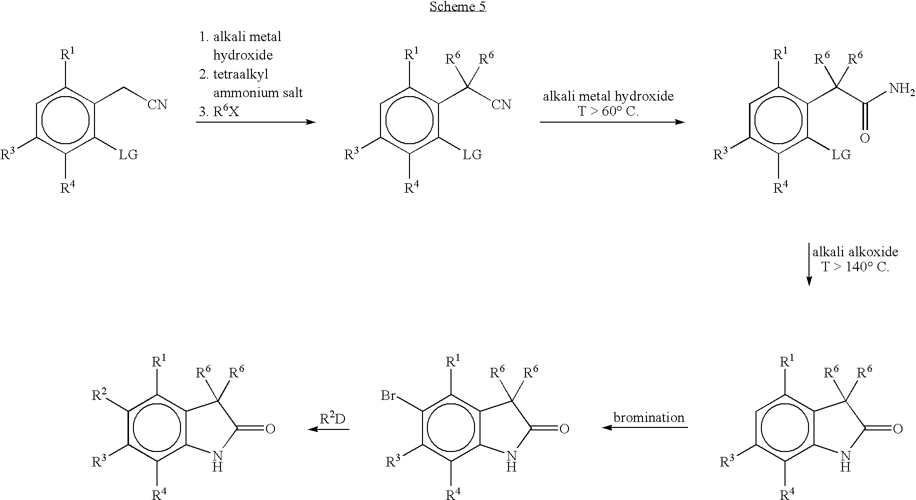

In a further embodiment, a process for preparing a substituted oxindol-2-one is provided and includes (i) reacting a first alkali metal hydroxide, a tetraalkylammonium salt, a benzonitrile, and XCH₂(CH₂)ₙX′, wherein n is 1 to 5 and X and X′ are leaving groups; (ii) reacting the product of step (i) with a second alkali metal hydroxide at a temperature of at least about 60° C.; (iii) reacting the product of step (ii) with an alkali alkoxide at a temperature of at least about 140° C. to form an oxindol-2-one; (iv) brominating the oxindol-2-one; and (v) coupling the brominated oxindol-2-one with a coupling reagent. See, Scheme 6.

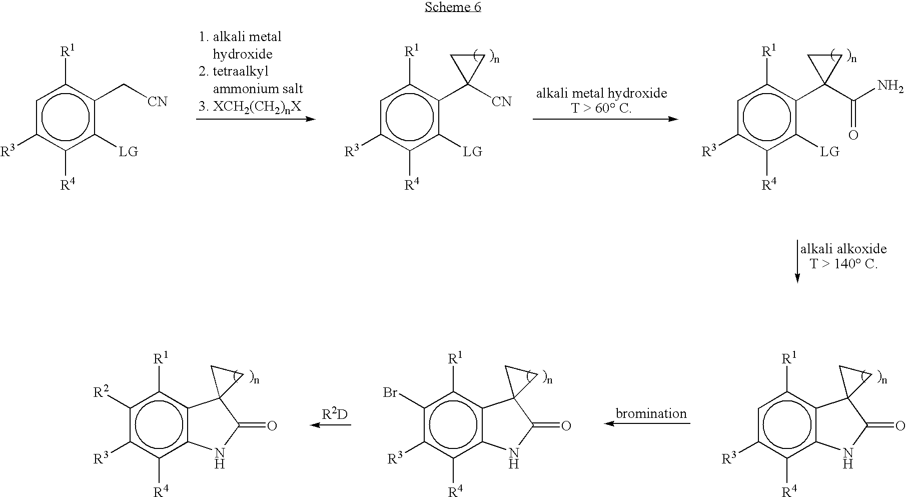

In another embodiment, a process for preparing a compound of the following structure is provided.

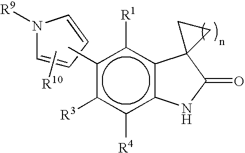

wherein, R¹, R³, and R⁴ are, independently, selected from among H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein the C₂ to C₆ alkynyl and substituted C₂ to C₆ alkynyl groups contain internal triple bonds; or R¹ and R³; R³ and R⁴; or R¹, R³, and R⁴ are fused to form (a) a 3 to 15 membered fully saturated, partially saturated, or fully unsaturated carbon-containing ring; or (b) a 3 to 15 membered heterocyclic ring containing in its backbone from 1 to 3 heteroatoms selected from among O, S, and NR¹¹; and R⁵ is selected from among C₁ to C₆ alkyl and C₁ to C₆ substituted alkyl; R⁹ is selected from among C₁ to C₆ alkyl, substituted C₁ to C₆ alkyl, aryl, substituted aryl, heteroaryl, substituted heteroaryl, and COR^{A}; R^{A} is selected from among H, C₁ to C₆ alkyl, substituted C₁ to C₆ alkyl, C₁ to C₆ alkoxy, substituted C₁ to C₆ alkoxy, C₁ to C₆ aminoalkyl, and substituted C₁ to C₆ aminoalkyl; R¹⁰ is selected from among H, OH, NH₂, CN, halogen, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₁ to C₆ alkoxy, substituted C₁ to C₆ alkoxy, C₁ to C₆ aminoalkyl, substituted C₁ to C₆ aminoalkyl, and COR^{A}; and R¹¹ is absent, H, C₁ to C₆ alkyl, substituted C₁ to C₆ alkyl, aryl, or substituted aryl. The process includes (i) reacting an alkali metal hydroxide, a catalytic amount of a tetraalkyl ammonium salt, XCH₂(CH₂)ₙX′, wherein X and X′ are halogen and n is 1 to 5, and a benzonitrile; (ii) reacting the product of step (i) with an alkali metal hydroxide at a temperature of at least about 60° C.; (iii) reacting the product of step (ii) with an alkali alkoxide at a temperature of at least about 140° C. to form an oxindol-2-one; (iv) brominating the oxindol-2-one; and (v) coupling the brominated oxindol-2-one with a coupling reagent.

In a further embodiment, a process for preparing the following compound is provided and includes reacting sodium hydroxide, a catalytic amount of tetrabutyl ammonium bromide, dibromoethane, and 2,3-difluoro-phenylacetonitrile; (ii) reacting the product of step (i) with potassium hydroxide at a temperature of at least about 60° C.; (iii) reacting the product of step (ii) with sodium t-pentoxide at a temperature of at least about 140° C.; (iv) brominating the product of step (iii); and (v) reacting the product of step (iv) with 5-[1,3,6,2]dioxazaborocan-2-yl-1-methyl-1H-pyrrole-2-carbonitrile. In one example, the bromination is performed using N-bromosuccinimide. In another example, the product of step (iii) is 4′-fluorospiro[cyclopropane-1,3]-indolin]-2′-one. In a further example, the product of step (iv) is 5′-bromo-4′-fluorospiro[cyclopropane-1,3′-indolin]-2′-one.

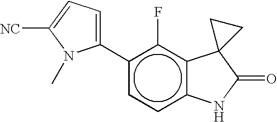

In still a further embodiment, a process for preparing a substituted oxindol-2-one is described and includes adding 2,6-difluoro-phenylacetonitrile to a mixture of 50% NaOH solution, about 0.04 equivalents of Bu₄NBr, and 2 equivalents of BrCH₂CH₂Br. See, Scheme 7. The temperature is maintained between about 35 to about 45° C. and, after addition, the reaction temperature is adjusted to about 45° C. and stirred for about 2 hours. Upon completion, water is slowly added, the reaction mixture cooled to about 20 to about 25° C., and MTBE is added to extract the product. The organic phase is then separated, concentrated by vacuum distillation and chased with t-amyl alcohol to remove water and unreacted BrCH₂CH₂Br and then the residue is dissolved into t-amyl alcohol. KOH is then added to this mixture, the reaction mixture heated to 70° C. and stirred for 1 hour. Upon completion, the reaction mixture is cooled 30° C., water is added, and the reaction mixture stirred for about 15 minutes. The organic phase is separated and concentrated by vacuum distillation. The residue of the reaction mixture is chased with toluene to remove water residue and t-amyl alcohol, and then dissolved into NMP. Sodium t-pentoxide is then added to the NMP solution, the reaction mixture heated to about 145° C. and stirred for about 4 to about 9 hours. The reaction mixture is then cooled to room temperature and added into a cold HCl solution slowly over 1 hour while maintaining the temperature between about 10 to about 20° C. The mixture is then cooled to about 3 to about 7° C. and stirred for about 1 hour. The solid is filtered and washed with water three times. The solid is dried at 55° C. under vacuum to give 4′-fluorospiro[cyclopropane-1,3′-indolin]-2′-one. N-bromosuccinimide in 3 parts of water is added to a slurry of 4′-fluorospiro[cyclopropane-1,3′-indolin]-2′-one in a mixture of 3 parts of water and 3 parts of acetonitrile at about 20 to about 25° C. over 1 hour. After addition, the reaction is stirred for 1 hour at about 20 to about 25° C. Upon completion, 3 parts of water is added, the mixture cooled to about 0 to about 6° C., the mixture stirred for 30 minutes, the solid filtered, the solid washed with water and dried at 55° C. for 18 hours at 0-10 mm Hg. The brominated 2-oxindole is then coupled using at least a 1% mol equivalent of dichloro bis(triphenylphosphine) palladium (II), about 1.2 equivalents of a weak base such as sodium or potassium carbonate, and [1,3,6,2]dioxazaborcan-2-yl-1-methyl-1H-pyrrole-2-carbonitrile in THF at a temperature of about 66° C. The substituted oxindol-2-one is then extracted in THF, about 0.5 equivalents of N-acetyl-L-cysteine added, the organic layer is refluxed with the N-acetyl-cysteine solution for about 1 hour, and filtered through a Celite® reagent/charco/Celite® reagent pad. The solvent is replaced with 2-propanol by vacuum distillation. The coupled compound is then crystallized, the solid isolated using filtration, the collected solid washed with cold 2-propanol, and the washed solid dried at about 45 to about 55° C. under vacuum to give the crude compound. The crude solid is then slurried in ethyl acetate at about 45 to about 50° C. for about 2 to about 3 hours, cooled to about 5 to about 10° C., the solid filtered and washed with cold ethyl acetate, and the final substituted oxindol-2-one dried using reduced pressures. See, Scheme 7.

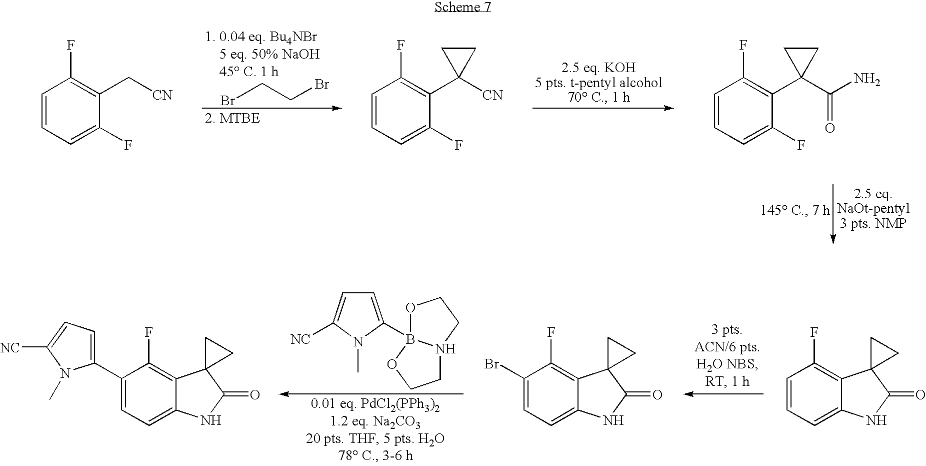

The following examples are illustrative only and are not intended to be a limitation on the present invention.

### EXAMPLES

### Example 1

### Preparation of 4′-fluorospiro[cyclopropane-1,3′-indolin]-2′-one

2,6-difluoro-phenylacetonitrile (306 g, 2.0 mol) was added to a mixture of 50% NaOH (800 g, 10 mol) solution, catalytic amount of Bu₄NBr (25.8 g, 0.08 mol) and BrCH₂CH₂Br (750 g, 4.0 mol) over 90 minutes. The temperature of the reaction mixture increased and the temperature was maintained at 35-45° C. After addition, the reaction temperature was adjusted to 45±2° C. and stirred for 2 hours. Water was then slowly added, the reaction mixture was cooled to 20-25° C., and methyl t-butyl ether (2000 mL) was added. The organic phase was separated, concentrated by vacuum distillation and chased with t-amyl alcohol (612 ml) to remove water and unreacted BrCH₂CH₂Br. The residue was dissolved into t-amyl alcohol (1530 mL).

Potassium hydroxide (KOH; 281 g, 5.0 mol) was added to this mixture, the reaction mixture was heated to 70±2° C., and the mixture was stirred for 1 hour. Upon completion, the reaction mixture was cooled 30±5° C., water (1530 g) was added, and the solution was stirred for 15 minutes. The organic phase was separated and concentrated by vacuum distillation. The residue of the reaction mixture was chased with toluene (2×306 mL) to remove water residue and t-amyl alcohol, and then dissolved into NMP (918 mL).

Sodium t-pentoxide (550 g, 5.0 mol) was added to the NMP solution, the mixture was heated to 145±2° C., and was stirred for 4-9 hours. Upon completion, the reaction mixture was cooled to room temperature and then added to a cold HCl solution (12 M, 416 mL, 5 mol) in water (3000 mL) slowly over 1 hour while the temperature was maintained between 10-20° C. A solid formed and the pH of the solution was 1-4. The mixture was cooled 3-7° C. and stirred for 1 hour. The solid was collected by filtration and washed with water (3×612 mL). The solid was dried at 55° C. under vacuum to give 4′-fluorospiro[cyclopropane-1,3′-indolin]-2′-one (254 g) in 72% yield with 98% HPLC purity, ¹H NMR (CDCl₃): δ 8.95 (s, 1H), 7.14 (m, 1H), 6.81 (d, 1H, J=7.7 Hz), 6.67 (t, 1H, J=9.0 Hz), 1.94 (m, 2H), 1.72 (m, 2H).

### Example 2

### Preparation of 5′-bromo-4′-fluorospiro[cyclopropane-1,3′-indolin]-2′-one

A slurry of N-bromosuccinimide (254 g, 1.43 mol) in 3 parts of water (798 mL) was added to a slurry of 4′-fluorospiro[cyclopropane-1,3′-indolin]-2′-one (266 g, 1.50 mol) in a mixture of 3 parts of water (798 mL) and 3 parts of acetonitrile (798 mL) at 20-25° C. over 1 hour. A slight exothermic reaction was observed. After addition, the reaction mixture was stirred for 1 hour at 20-25° C. Upon completion, 3 parts of water (798 mL) was added, the mixture was cooled to 0-6° C., and the mixture was stirred for 30 minutes. The solid was collected by filtration, washed with water, and dried at 55° C./18 hours/10 mm Hg to give the product (350 g) in 94% yield. ¹H NMR (CDCl₃): δ 8.79 (s, 1H), 7.38 (m, 1H), 6.71 (d, 1H, J=8.3 Hz), 1.96 (m, 2H), 1.78 (m, 2H).

### Example 3

### 5-(4′-fluoro-2′-oxo-1′,2′-dihydrospiro[cyclopropane-1,3′-indol]-5′-yl)-1-methyl-1H-pyrrole-2-carbonitrile

A mixture of 5′-bromo-4′-fluorospiro[cyclopropane-1,3′-indol]-2′(1′H)-one (10.0 g, 39.1 mmol), 5-[1,3,6,2]dioxazaborocan-2-yl-1-methyl-1H-pyrrole-2-carbonitrile (4.30 g, 19.5 mmol), Na₂CO₃ (4.97 g, 46.9 mmol), acetonitrile (150 mL), water (50 mL), and PdCl₂(PPh₃)₂ (0.301 g, 0.391 mmol) was heated to reflux for 1 hour. 5-[1,3,6,2]dioxazaborocan-2-yl-1-methyl-1H-pyrrole-2-carbonitrile (12.8 g, 58.4 mmol) was then added in three portions every hour. The mixture was heated to reflux for 1 hour and then the reaction mixture was cooled to room temperature. The organic solvent was removed by distillation. The crude solid was filtered, washed with a mixture of water and acetonitrile (1:1, v/v, 3×10 mL), and dried to give a crude solid.

The crude solid was dissolved in ethylacetate (EtOAc; 60 mL) at reflux. A solution of N-acetyl-L-cysteine (0.64 g, 0.391 mmol) in water (15 mL) was added and the mixture was heated to 70° C. for 2 hours. The organic layer was separated, charcoal (1 g) was added, and the solution refluxed for 2 hour. The mixture was filtered through a glass-sintered funnel packed with a pad of the Celite® reagent (10 g) and the pad was washed with EtOAc (2×10 mL). The filtrate was cooled to 0-5° C. The solid was collected by filtration, washed with EtOAc (2×5 mL), and dried using vacuum to give a white solid (6.0 g, 54% yield with 99% HPLC purity). ¹H NMR (DMSO-d₆): δ 9.50 (s, 1H), 7.25 (t, 1H, J=7.7 Hz), 7.04 (d, 1H, J=4 Hz), 6.88 (d, 1H, J=8 Hz), 6.27 (d, 1H, J=4 Hz), 3.58 (s, 3H), 1.85 (m, 2H), 1.49 (m, 2H).

### Example 4

### Alternative Preparation of 5-(4′-fluoro-2′-oxo-1′,2′-dihydrospiro[cyclopropane-1,3′-indol]-5′-yl)-1-methyl-1H-pyrrole-2-carbonitrile

5′-bromo-4′-fluorospiro[cyclopropane-1,3′-indol]-2′(1′H)-one (100 g, 0.391 mol), PdCl₂(PPh₃)₂ (2.74 g, 0.0033 mol), 5-[1,3,6,2]dioxazaborocan-2-yl-1-methyl-1H-pyrrole-2-carbonitrile (43 g, 0.196 mol) and THF (1500 mL) were added to a 2 L reactor. A sodium carbonate solution (49.7 g, 0.469 mol) in water (450 mL) was charged to the reactor. The resulting mixture was then heated to and held at 70-75° C. After 45 minutes, analysis of the mixture by high performance liquid chromatography (HPLC) indicated that the reaction had initiated. After 1 hour, 5-[1,3,6,2]dioxazaborocan-2-yl-1-methyl-1H-pyrrole-2-carbonitrile (64 g, 0.292 mol) was added. Thirty minutes after the addition, HPLC indicated that the reaction was progressing. Additional 5-[1,3,6,2]dioxazaborocan-2-yl-1-methyl-1H-pyrrole-2-carbonitrile (64 g, 0.292 mol) was then added. One hour after the addition, HPLC indicated that the reaction was complete. Heating was discontinued and the reaction mixture was cooled to 20-25° C. An aqueous solution sodium chloride (sodium chloride 126 g in water 450 mL) was added to the stirred mixture. The aqueous layer was removed and N-acetyl-L-cysteine (32 g, 0.196 mol) in THF solution was added to the organic layer. The mixture was heated and stirred to a gentle reflux temperature of 60-65° C. for a minimum of 1 hour. After cooling to 35-45° C., the mixture was filtered over a pad containing the Celite® reagent/the Darco® reagent/the Celite® reagent (66 g/70 g/66 g) through a sintered glass filter. The filter cake was washed with THF (2×300 mL). The combined filtrate and washes were concentrated by vacuum distillation. THF was replaced with 2-propanol (1.0 L) using continued concentration to remove THF to a residual volume of about 575 mL (target THF <5%, target moisture <1%). The slurry was cooled to 10-15° C. and seeded. After stirring for 0.5 hours, the slurry was filtered through a sintered glass filter funnel. The solid was washed with cold 2-propanol, slurried in ethyl acetate (125 mL) at 45-50° C. for 2-3 hours, cooled to 5-10° C., and filtered and washed with cold ethyl acetate (2×20 mL). The product was dried under reduced pressures for several hours to give the product (21.3 g) as a light off-white solid with 99.03% HPLC purity.

All publications cited in this specification are incorporated herein by reference. While the invention has been described with reference to particular embodiments, it will be appreciated that modifications can be made without departing from the spirit of the invention. Such modifications are intended to fall within the scope of the appended claims.

What is claimed is:

1. A process for preparing a substituted oxindol-2-one, said process comprising:
(i) reacting a first alkali metal hydroxide, a tetraalkyl ammonium salt, a benzonitrile, and R⁶X or XCH₂(CH₂)ₙX′, wherein:
R⁶ is C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, heterocyclic, or substituted heterocyclic;
X and X′ are, independently, leaving groups; and
n is 1 to 5;
(ii) reacting the product of step (i) with a second alkali metal hydroxide at a temperature of at least about 60° C.;
(iii) reacting the product of step (ii) with an alkali alkoxide at a temperature of at least about 140° C. to form an oxindol-2-one;
(iv) brominating said oxindol-2-one; and
(v) coupling the brominated oxindol-2-one with a coupling reagent.

2. The process according to claim 1, wherein step (ii) is performed at about 60 to about 100° C.

3. The process according to claim 1, wherein step (iii) is performed at about 140 to about 180° C.

4. The process according to claim 1, wherein said substituted oxindol-2-one is of the structure: 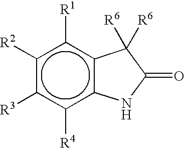
wherein:
R¹, R³, and R⁴ are, independently, selected from the group consisting of H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein said C₂ to C₆ alkynyl and substituted C₂ to C₆ alkynyl groups comprise internal triple bonds; or
R¹ and R³; R³ and R⁴; or R¹, R³, and R⁴ are fused to form:
(a) a 3 to 15 membered fully saturated, partially saturated, or fully unsaturated carbon-containing ring; or
(b) a 3 to 15 membered heterocyclic ring containing in its backbone from 1 to 3 heteroatoms selected from the group consisting of O, S, and NR¹¹;
R² is C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, heterocyclic, or substituted heterocyclic;
R⁵ is selected from the group consisting of C₁ to C₆ alkyl and C₁ to C₆ substituted alkyl;
R⁶ is C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, heterocyclic, or substituted heterocyclic; and
R¹¹ is absent, H, C₁ to C₆ alkyl, substituted C₁ to C₆ alkyl, aryl, or substituted aryl.

5. The process according to claim 1, wherein said substituted oxindol-2-one is of the structure: 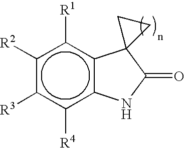
wherein:
R¹, R³, and R⁴ are, independently, selected from the group consisting of H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein said C₂ to C₆ alkynyl and substituted C₂ to C₆ alkynyl groups comprise internal triple bonds; or
R¹ and R³; R³ and R⁴; or R¹, R³, and R⁴ are fused to form:
(a) a 3 to 15 membered fully saturated, partially saturated, or fully unsaturated carbon-containing ring; or
(b) a 3 to 15 membered heterocyclic ring containing in its backbone from 1 to 3 heteroatoms selected from the group consisting of O, S, and NR¹¹;
R² is C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, heterocyclic, or substituted heterocyclic;
R⁵ is selected from the group consisting of C₁ to C₆ alkyl and C₁ to C₆ substituted alkyl; and
R¹¹ is absent, H, C₁ to C₆ alkyl, substituted C₁ to C₆ alkyl, aryl, or substituted aryl.

6. The process according to claim 1, wherein said substituted oxindol-2-one is of the structure: 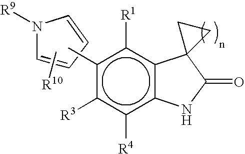
wherein:
R¹, R³, and R⁴ are, independently, selected from the group consisting of H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein said C₂ to C₆ alkynyl and substituted C₂ to C₆ alkynyl groups comprise internal triple bonds; or
R¹ and R³; R³ and R⁴; or R¹, R³, and R⁴ are fused to form:
(a) a 3 to 15 membered fully saturated, partially saturated, or fully unsaturated carbon-containing ring; or
(b) a 3 to 15 membered heterocyclic ring containing in its backbone from 1 to 3 heteroatoms selected from the group consisting of O, S, and NR¹¹; and
R⁵ is selected from the group consisting of C₁ to C₆ alkyl and C₁ to C₆ substituted alkyl;
R⁹ is selected from the group consisting of C₁ to C₆ alkyl, substituted C₁ to C₆ alkyl, aryl, substituted aryl, heteroaryl, substituted heteroaryl, and COR^{A};
R^{A} is selected from the group consisting of H, C₁ to C₆ alkyl, substituted C₁ to C₆ alkyl, C₁ to C₆ alkoxy, substituted C₁ to C₆ alkoxy, C₁ to C₆ aminoalkyl, and substituted C₁ to C₆ aminoalkyl;
R¹⁰ is selected from the group consisting of H, OH, NH₂, CN, halogen, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₁ to C₆ alkoxy, substituted C₁ to C₆ alkoxy, C₁ to C₆ aminoalkyl, substituted C₁ to C₆ aminoalkyl, and COR^{A}; and
R¹¹ is absent, H, C₁ to C₆ alkyl, substituted C₁ to C₆ alkyl, aryl, or substituted aryl.

7. The process according to claim 1, wherein said substituted oxindol-2-one is of the structure: 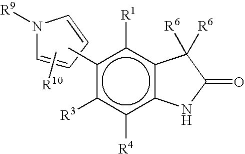
wherein:
R¹, R³, and R⁴ are, independently, selected from the group consisting of H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein said C₂ to C₆ alkynyl and substituted C₂ to C₆ alkynyl groups comprise internal triple bonds; or
R¹ and R³; R³ and R⁴; or R¹, R³, and R⁴ are fused to form:
(a) a 3 to 15 membered fully saturated, partially saturated, or fully unsaturated carbon-containing ring; or
(b) a 3 to 15 membered heterocyclic ring containing in its backbone from 1 to 3 heteroatoms selected from the group consisting of O, S, and NR¹¹; and
R⁵ is selected from the group consisting of C₁ to C₆ alkyl and C₁ to C₆ substituted alkyl;
R⁶ is C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, heterocyclic, or substituted heterocyclic;
R⁹ is selected from the group consisting of C₁ to C₆ alkyl, substituted C₁ to C₆ alkyl, aryl, substituted aryl, heteroaryl, substituted heteroaryl, and COR^{A};
R^{A} is selected from the group consisting of H, C₁ to C₆ alkyl, substituted C₁ to C₆ alkyl, C₁ to C₆ alkoxy, substituted C₁ to C₆ alkoxy, C₁ to C₆ aminoalkyl, and substituted C₁ to C₆ aminoalkyl;
R¹⁰ is selected from the group consisting of H, OH, NH₂, CN, halogen, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₁ to C₆ alkoxy, substituted C₁ to C₆ alkoxy, C₁ to C₆ aminoalkyl, substituted C₁ to C₆ aminoalkyl, and COR^{A}; and
R¹¹ is absent, H, C₁ to C₆ alkyl, substituted C₁ to C₆ alkyl, aryl, or substituted aryl.

8. The process according to claim 1, wherein said substituted oxindol-2-one is 5-(4′-fluoro-2′-oxo-1′,2′-dihydrospiro[cyclopropane-1,3′-indol]-5′-yl)-1-methyl-1H-pyrrole-2-carbonitrile.

9. The process according to claim 1, wherein said benzonitrile is of the structure: 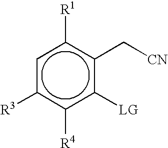
wherein:
R¹, R³, and R⁴ are, independently, selected from the group consisting of H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein said C₂ to C₆ alkynyl and substituted C₂ to C₆ alkynyl groups comprise internal triple bonds;
or R³ and R⁴ are fused to form:
(a) a 3 to 15 membered fully saturated, partially saturated, or fully unsaturated carbon-containing ring; or
(b) a 3 to 15 membered heterocyclic ring containing in its backbone from 1 to 3 heteroatoms selected from the group consisting of O, S, and NR¹¹;
R⁵ is selected from the group consisting of C₁ to C₆ alkyl and C₁ to C₆ substituted alkyl;
LG is a leaving group; and
R¹¹ is absent, H, C₁ to C₆ alkyl, substituted C₁ to C₆ alkyl, aryl, or substituted aryl.

10. The process according to claim 1, wherein the product of step (i) is of the structure: 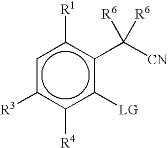
wherein:
R¹, R³, and R⁴ are, independently, selected from the group consisting of H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein said C₂ to C₆ alkynyl and substituted C₂ to C₆ alkynyl groups comprise internal triple bonds;
or R³ and R⁴ are fused to form:
(a) a 3 to 15 membered fully saturated, partially saturated, or fully unsaturated carbon-containing ring; or
(b) a 3 to 15 membered heterocyclic ring containing in its backbone from 1 to 3 heteroatoms selected from the group consisting of O, S, and NR¹¹;
R⁵ is selected from the group consisting of C₁ to C₆ alkyl and C₁ to C₆ substituted alkyl;
R⁶ is C₁ to C₆ alkyl substituted C₁ to C₆ alkyl, C₃ to C₈ cycloalkyl, substituted C₃ to C₈ cycloalkyl, aryl, substituted aryl, heteroaryl, substituted heteroaryl, heterocyclic, or substituted heterocyclic;
LG is a leaving group; and
R¹¹ is absent, H, C₁ to C₆ alkyl, substituted C₁ to C₆ alkyl, aryl, or substituted aryl.

11. The process according to claim 1, wherein the product of step (i) is of the structure: 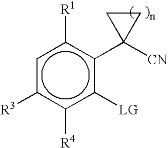
wherein:
R¹, R³, and R⁴ are, independently, selected from the group consisting of H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein said C₂ to C₆ alkynyl and substituted C₂ to C₆ alkynyl groups comprise internal triple bonds;
or R³ and R⁴ are fused to form:
(a) a 3 to 15 membered fully saturated, partially saturated, or fully unsaturated carbon-containing ring; or
(b) a 3 to 15 membered heterocyclic ring containing in its backbone from 1 to 3 heteroatoms selected from the group consisting of O, S, and NR¹¹;
R⁵ is selected from the group consisting of C₁ to C₆ alkyl and C₁ to C₆ substituted alkyl;
R¹¹ is absent, H, C₁ to C₆ alkyl, substituted C₁ to C₆ alkyl, aryl, or substituted aryl; and
LG is a leaving group.

12. The process according to claim 1, wherein the product of step (ii) is of the structure: 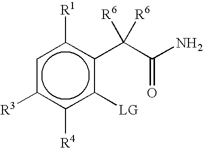
R¹, R³, and R⁴ are, independently, selected from the group consisting of H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein said C₂ to C₆ alkynyl and substituted C₂ to C₆ alkynyl groups comprise internal triple bonds;
or R³ and R⁴ are fused to form:
(a) a 3 to 15 membered fully saturated, partially saturated, or fully unsaturated carbon-containing ring; or
(b) a 3 to 15 membered heterocyclic ring containing in its backbone from 1 to 3 heteroatoms selected from the group consisting of O, S, and NR¹¹;
R⁵ is selected from the group consisting of C₁ to C₆ alkyl and C₁ to C₆ substituted alkyl;
R⁶ is C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, heterocyclic, or substituted heterocyclic;
R¹¹ is absent, H, C₁ to C₆ alkyl, substituted C₁ to C₆ alkyl, aryl, or substituted aryl; and
LG is a leaving group.

13. The process according to claim 1, wherein the product of step (ii) is of the structure: 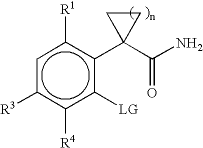
R¹, R³, and R⁴ are, independently, selected from the group consisting of H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein said C₂ to C₆ alkynyl and substituted C₂ to C₆ alkynyl groups comprise internal triple bonds;
or R³ and R⁴ are fused to form:
(a) a 3 to 15 membered fully saturated, partially saturated, or fully unsaturated carbon-containing ring; or
(b) a 3 to 15 membered heterocyclic ring containing in its backbone from 1 to 3 heteroatoms selected from the group consisting of O, S, and NR¹¹;
R⁵ is selected from the group consisting of C₁ to C₆ alkyl and C₁ to C₆ substituted alkyl;
R¹¹ is absent, H, C₁ to C₆ alkyl, substituted C₁ to C₆ alkyl, aryl, or substituted aryl; and
LG is a leaving group.

14. The process according to claim 1, wherein the product of step (iii) is of the structure: 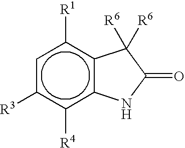
wherein:
R¹, R³, and R⁴ are, independently, selected from the group consisting of H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein said C₂ to C₆ alkynyl and substituted C₂ to C₆ alkynyl groups comprise internal triple bonds;
or R³ and R⁴ are fused to form:
(a) a 3 to 15 membered fully saturated, partially saturated, or fully unsaturated carbon-containing ring; or
(b) a 3 to 15 membered heterocyclic ring containing in its backbone from 1 to 3 heteroatoms selected from the group consisting of O, S, and NR¹¹;
R⁵ is selected from the group consisting of C₁ to C₆ alkyl and C₁ to C₆ substituted alkyl;
R⁶ is C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, heterocyclic, or substituted heterocyclic; and
R¹¹ is absent, H, C₁ to C₆ alkyl, substituted C₁ to C₆ alkyl, aryl, or substituted aryl.

15. The process according to claim 1, wherein the product of step (iii) is of the structure: 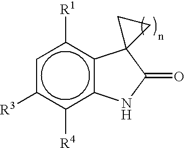
wherein:
R¹, R³, and R⁴ are, independently, selected from the group consisting of H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein said C₂ to C₆ alkynyl and substituted C₂ to C₆ alkynyl groups comprise internal triple bonds;
or R³ and R⁴ are fused to form:
(a) a 3 to 15 membered fully saturated, partially saturated, or fully unsaturated carbon-containing ring; or
(b) a 3 to 15 membered heterocyclic ring containing in its backbone from 1 to 3 heteroatoms selected from the group consisting of O, S, and NR¹¹;
R⁵ is selected from the group consisting of C₁ to C₆ alkyl and C₁ to C₆ substituted alkyl; and
R¹¹ is absent, H, C₁ to C₆ alkyl, substituted C₁ to C₆ alkyl, aryl, or substituted aryl.

16. The process according to claim 1, wherein the product of step (iii) is 4′-fluorospiro[cyclopropane-1,3′-indolin]-2′-one.

17. The process according to claim 1, wherein the product of step (iv) is of the structure: 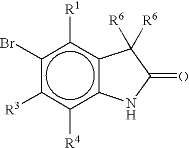
wherein:
R¹, R³, and R⁴ are, independently, selected from the group consisting of H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein said C₂ to C₆ alkynyl and substituted C₂ to C₆ alkynyl groups comprise internal triple bonds;
or R³ and R⁴ are fused to form:
(a) a 3 to 15 membered fully saturated, partially saturated, or fully unsaturated carbon-containing ring; or
(b) a 3 to 15 membered heterocyclic ring containing in its backbone from 1 to 3 heteroatoms selected from the group consisting of O, S, and NR¹¹;
R⁵ is selected from the group consisting of C₁ to C₆ alkyl and C₁ to C₆ substituted alkyl;
R⁶ is C₁ to C₆ alkyl, substituted C₁ to C₆ alkyl, C₃ to C₈ cycloalkyl, substituted C₃ to C₈ cycloalkyl, aryl, substituted aryl, heteroaryl, substituted heteroaryl, heterocyclic, or substituted heterocyclic; and
R¹¹ is absent, H, C₁ to C₆ alkyl, substituted C₁ to C₆ alkyl, aryl, or substituted aryl.

18. The process according to claim 1, wherein the product of step (iv) is of the structure: 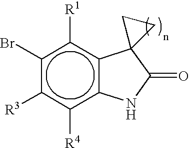
wherein:
R¹, R³, and R⁴ are, independently, selected from the group consisting of H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein said C₂ to C₆ alkynyl and substituted C₂ to C₆ alkynyl groups comprise internal triple bonds;
or R³ and R⁴ are fused to form:
(a) a 3 to 15 membered fully saturated, partially saturated, or fully unsaturated carbon-containing ring; or
(b) a 3 to 15 membered heterocyclic ring containing in its backbone from 1 to 3 heteroatoms selected from the group consisting of O, S, and NR¹¹;
R⁵ is selected from the group consisting of C₁ to C₆ alkyl and C₁ to C₆ substituted alkyl; and
R¹¹ is absent, H, C₁ to C₆ alkyl, substituted C₁ to C₆ alkyl, aryl, or substituted aryl.

19. The process according to claim 1, wherein the product of step (iv) is 5′-bromo-4′-fluorospiro[cyclopropane-1,3′-indolin]-2′-one.

20. The process according to claim 1, wherein said coupling reagent is[1,3,6,2]dioxazaborocan-2-yl-1-methyl-1H-pyrrole-2-carbonitrile.

21. The process according to claim 1, wherein said coupling is performed in the presence of a palladium catalyst and said process further comprises extracting said substituted oxindol-2-one using an organic solvent, removing said palladium catalyst by adding N-acetyl-L-cysteine to said organic solvent comprising said substituted oxindol-2-one, and filtering said organic solvent comprising said substituted oxindol-2-one.

22. The process according to claim 21, further comprising removing said organic solvent to provide a crude solid, dissolving said crude solid in 2-propanol, and recrystallizing said substituted oxindol-2-one.

23. A process for preparing a compound of the structure: 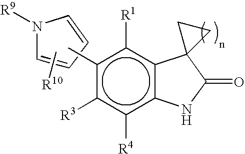
wherein:
R¹, R³, and R⁴ are, independently, selected from the group consisting of H, chlorine, CN, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₃ to C₈ cycloalkyl, substituted C₃ to C₈ cycloalkyl, aryl, substituted aryl, heteroaryl, substituted heteroaryl, OSO₂CF₃, CF₃, NO₂, SR⁵, OR⁵, N(R⁵)₂, COOR⁵, CON(R⁵)₂, and SO₂N(R⁵)₂, wherein said C₂ to C₆ alkynyl and substituted C₂ to C₆ alkynyl groups comprise internal triple bonds;
R¹ and R³; R³ and R⁴; or R¹, R³, and R⁴ are fused to form:
(a) a 3 to 15 membered fully saturated, partially saturated, or fully unsaturated carbon-containing ring; or
(b) a 3 to 15 membered heterocyclic ring containing in its backbone from 1 to 3 heteroatoms selected from the group consisting of O, S, and NR¹¹; and
R⁵ is selected from the group consisting of C₁ to C₆ alkyl and C₁ to C₆ substituted alkyl;
R⁹ is selected from the group consisting of C₁ to C₆ alkyl, substituted C₁ to C₆ alkyl, aryl, substituted aryl, heteroaryl, substituted heteroaryl, and COR^{A};
R^{A} is selected from the group consisting of H, C₁ to C₆ alkyl, substituted C₁ to C₆ alkyl, C₁ to C₆ alkoxy, substituted C₁ to C₆ alkoxy, C₁ to C₆ aminoalkyl, and substituted C₁ to C₆ aminoalkyl;
R¹⁰ is selected from the group consisting of H, OH, NH₂, CN, halogen, C₁ to C₆ alkyl, substituted C₁ to C₆ alkyl, C₂ to C₆ alkenyl, substituted C₂ to C₆ alkenyl, C₂ to C₆ alkynyl, substituted C₂ to C₆ alkynyl, C₁ to C₆ alkoxy, substituted C₁ to C₆ alkoxy, C₁ to C₆ aminoalkyl, substituted C₁ to C₆ aminoalkyl, and COR^{A}; and
R¹¹ is absent, H, C₁ to C₆ alkyl, substituted C₁ to C₆ alkyl, aryl, or substituted aryl; said process comprising:
(i) reacting an alkali metal hydroxide, a catalytic amount of a tetraalkyl ammonium salt, XCH₂(CH₂)ₙX′, wherein X and X′ are halogen and n is 1 to 5, and a benzonitrile;
(ii) reacting the product of step (i) with an alkali metal hydroxide at a temperature of at least about 60° C.;
(iii) reacting the product of step (ii) with an alkali alkoxide at a temperature of at least about 140° C. to form an oxindol-2-one;
(iv) brominating said oxindol-2-one; and
(v) coupling the brominated oxindol-2-one with a coupling reagent.

24. A process for preparing a compound of the structure: 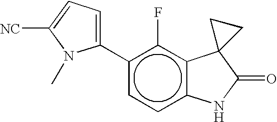
said process comprising:
(i) reacting sodium hydroxide, a catalytic amount of tetrabutyl ammonium bromide, dibromoethane, and 2,3-difluoro-phenylacetonitrile;
(ii) reacting the product of step (i) with potassium hydroxide at a temperature of at least about 60° C.;
(iii) reacting the product of step (ii) with sodium t-pentoxide at a temperature of at least about 140° C.;
(iv) brominating the product of step (iii); and
(v) reacting the product of step (iv) with 5-[1,3,6,2]dioxazaborocan-2-yl-1-methyl-1H-pyrrole-2-carbonitrile.

25. The process according to claim 24, wherein the product of step (iii) is 4′-fluorospiro[cyclopropane-1,3]-indolin]-2′-one.

26. The process according to claim 24, wherein the product of step (iv) is 5′-bromo-4′-fluorospiro[cyclopropane-1,3′-indolin]-2′-one.
